(19) United States
(12) Patent Application Publication
(10) Pub. No.: US 2023/0145940 A1
(43) Pub. Date: May 11, 2023

(54) NEUROTOXIN COMPOSITIONS FOR USE IN TREATING CARDIOVASCULAR DISORDERS

(71) Applicant: AEON Biopharma, Inc., Irvine, CA (US)

(72) Inventors: Gregory F. Brooks, Irvine, CA (US); Adelbert L. Stagg, Irvine, CA (US)

(21) Appl. No.: 17/911,089

(22) PCT Filed: Mar. 12, 2021
(86) PCT No.: PCT/US2021/022223

§ 371 Date: Sep. 12, 2022

Related U.S. Application Data
(60) Provisional application No. 62/988,764, filed on Mar. 12, 2020.

(51) Int. Cl. A61K 38/48; A61P 9/12; A61P 9/06; A61P 9/10; A61K 31/167; A61P 9/04

(52) U.S. Cl. CPC A61K 38/4893; C12Y 304/24069; A61P 9/12; A61P 9/06; A61P 9/10; A61K 31/167; A61P 9/04

(57) ABSTRACT

Disclosed herein are compositions and methods for use in treating cardiovascular disorders.

### FIELD

The present specification relates to the use of neurotoxins administered to the Stellate Ganglion (SG) nerve collection, for example to treat cardiovascular disorders.

### BACKGROUND

The SG is a collection of nerves found at the level of the sixth and seventh cervical vertebrae (the last vertebra of the neck). These nerves are located in front of the vertebrae. They are part of the sympathetic nervous system, supplying the head, upper extremities, and organs of the chest. Treatment, for example by “blocking” or modulating the SG (establishing an “SGB”, or a “Stellate Ganglion Block”) can be useful in treating a number of disorders, including cardiovascular disorders or diseases, for example angina (such as intractable angina), arrhythmias, myocardial contractility disorder (congestive heart failure), coronary artery disease, high blood pressure, and combinations thereof.

The autonomic nervous system plays an important role in the regulation of blood pressure, heart rate, myocardial contractility and coronary perfusion, and the balance between its sympathetic and parasympathetic branches has a key part in this. An imbalance in the autonomic cardiac fibers can further lead, inter alia, to cardiac arrhythmias. In general, an imbalance of the sympathetic and parasympathetic branches of the autonomic nervous system can also cause pain and inflammation, both in the short and long term. The various patho-mechanisms involved can be influenced by injection of local anesthetics, for example by an SGB. Several authors in the past have demonstrated that SGB using local anesthetic has a beneficial effect on cardiac arrhythmias.

### SUMMARY

Disclosed herein are compositions and methods comprising neurotoxins, for example Clostridial neurotoxins including botulinum toxins, and the use thereof to treat cardiovascular diseases and disorders, for example myocardial contractility disorder, angina, arrhythmias, coronary artery disease, high blood pressure, and combinations thereof. Disclosed methods can comprise the use of both intra-muscular and nerve-rich administration sites, for example injection into the stellate ganglion to “block” or modulate the nerve bundle, establishing an SGB.

Disclosed treatment modalities can prevent, alleviate, or eliminate symptoms of cardiovascular disorders. Longer duration of effect and increased reduction in symptoms as compared to anesthetic treatment of the SG alone can also be provided by the disclosed methods. Disclosed treatment methods comprise use of a neurotoxin applied to the SG or the vicinity thereof or combinations thereof. Disclosed treatment methods comprise use of a neurotoxin in combination with or without a local anesthetic with both applied to the SG or the vicinity thereof. Disclosed combination treatments, for example, a neurotoxin in combination with a drug suitable for treating a cardiovascular disorder, can provide a synergistic effect as compared to the effects of either administered alone when treating cardiovascular disorders such as angina, arrhythmias, myocardial contractility disorder, coronary artery disease, high blood pressure, and combinations thereof.

Disclosed embodiments comprise use of a neurotoxin to establish an SGB and/or modulate the activity of the SG. Disclosed embodiments comprise administering a therapeutically effective amount of at least one neurotoxin into the SG or the vicinity thereof, or both. In embodiments comprising injection into the SG, suitable compositions can comprise Clostridial neurotoxins, for example botulinum neurotoxins.

Treatments disclosed herein can provide increased duration of relief as compared to current methods.

### BRIEF DESCRIPTION OF THE DRAWINGS

FIG. 1 shows the location of the Stellate Ganglion within the neck.

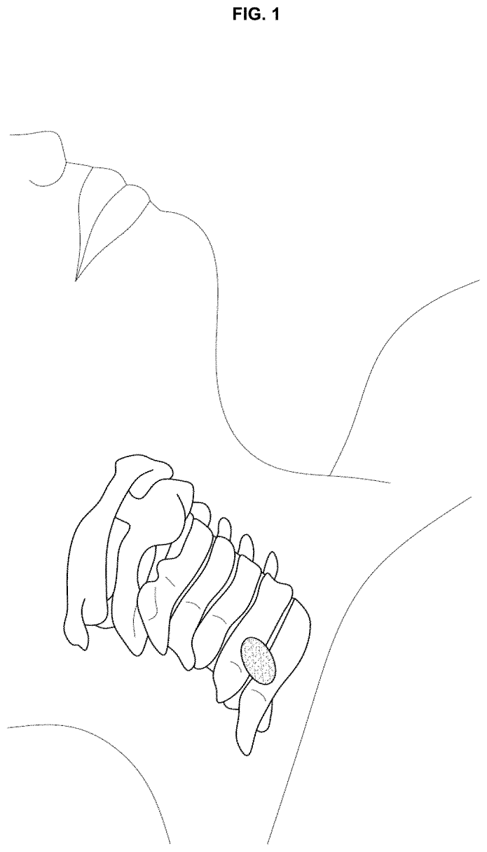

FIG. 2 shows a schematic of the neutral supine position of the neck.

FIG. 3 shows a schematic of the extended supine position of the neck.

### DETAILED DESCRIPTION

The present disclosure is directed toward methods for reducing the occurrence and severity of symptoms associated with cardiovascular disorders including intractable angina, arrhythmias, myocardial contractility, coronary artery disease, high blood pressure, and combinations thereof, for example through the use of a neurotoxin-induced or neurotoxin-mediated SG block, or SGB. SGB is a procedure selectively used by anesthesiologists to relieve pain. Emerging research suggests that SGB using a local anesthetic may help a subset of patients with cardiac disorders who have not found relief from traditional treatments such as medication.

Disclosed embodiments comprise use of a neurotoxin to establish an SGB or to modulate the activity of the SG. Disclosed embodiments comprise administering a therapeutically effective amount of at least one neurotoxin into the SG or the vicinity thereof. In embodiments comprising injection into the SG or vicinity thereof, suitable compositions can comprise Clostridial neurotoxins, for example botulinum neurotoxins. Disclosed embodiments comprise combination treatments wherein a drug useful for treating a cardiovascular disorder is also administered. For example, cardiac arrhythmias are often treated with drugs that come in pill form and are typically used long-term. In emergencies, some can be given intravenously.

Disclosed embodiments comprise the use of, for example, an arrhythmia drug in combination with a neurotoxin. For example, in disclosed embodiments, suitable arrhythmia drugs can include amiodarone, flecainide, ibutilide, lidocaine, procainamide, propafenone, quinidine and tocainide. Dosages of these drugs when used in disclosed methods can be reduced as compared to usage in the absence of a neurotoxin administration. Combinations of these drugs can also be used in disclosed embodiments.

In embodiments, the arrhythmia treatment drug can comprise a calcium channel blocker such as amlodipine, diltiazem, felodipine, isradipine, nicardipine, nifedipine nisoldipine and verapamil. Dosages of these drugs when used in disclosed methods can be reduced as compared to usage in the absence of a neurotoxin administration. Combinations of these drugs can also be used in disclosed embodiments. Calcium channel blockers can also be used in disclosed embodiments for treatment of angina.

In embodiments, the arrhythmia treatment drug can comprise a beta-blocker, which typically lessens or prevents the action of the hormone adrenaline, which can relieve tachycardia by slowing heart rate. Beta-blockers can also lower blood pressure and decrease the stress on the heart. Examples of beta blockers suitable for use in disclosed embodiments include, for example, acebutolol, atenolol, bisoprolol, metoprolol nadolol and propranolol. Dosages of these drugs when used in disclosed methods can be reduced as compared to usage in the absence of a neurotoxin administration. Combinations of these drugs can also be used in disclosed embodiments.

In embodiments, cardiac contractility can be treated by use of an inotropic agent such as milrinone, digoxin, dopamine, and dobutamine. Dosages of these drugs when used in disclosed methods can be reduced as compared to usage in the absence of a neurotoxin administration. Combinations of these drugs can also be used in disclosed embodiments.

In disclosed embodiments, disorders involving coronary artery disease can be treated, for example using nitrates, morphine, beta-blockers, calcium channel blockers, ranolazine and angiotensin-converting enzyme inhibitors, angiotensin II receptor blockers and combinations thereof. Dosages of these drugs when used in disclosed methods can be reduced as compared to usage in the absence of a neurotoxin administration.

Disclosed embodiments, comprise methods of treating high blood pressure with drugs including, for example, diuretics, beta-blockers. ACE inhibitors, Angiotensin II receptor blockers, calcium channel blockers, alpha blockers, alpha-2 receptor agonists and combinations thereof, for example combinations of alpha and beta-blockers. Dosages of these drugs when used in disclosed methods can be reduced as compared to usage in the absence of a neurotoxin administration.

### Definitions

“Administration,” or “to administer” means the step of giving (i.e. administering) a pharmaceutical composition or active ingredient to a subject. The pharmaceutical compositions disclosed herein can be administered via a number of appropriate routs, including oral and intramuscular or subcutaneous routes of administration, such as by injection, topically, or use of an implant.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Cardiovascular Disorder” or “Cardiovascular Disease” generally refers to disorders of the heart and surrounding tissue, including the circulatory system, including angina, arrhythmias, myocardial contractility, coronary artery disease, cardiomyopathy, high blood pressure, and the like, and combinations thereof.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Fast-acting neurotoxin” as used herein refers to a botulinum toxin that produces effects in the patient more rapidly than those produced by, for example, a botulinum neurotoxin type A. For example, the effects of a fast-acting botulinum toxin (such as botulinum type E) can be produced within 36 hours.

“Fast-recovery neurotoxin” as used herein refers to a botulinum toxin that whose effects diminish in the patient more rapidly than those produced by, for example, a botulinum neurotoxin type A. For example, the effects of a fast-recovery botulinum toxin (such as botulinum type E) can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months, and in some circumstances for as long as 27 months, when used to treat glands, such as in the treatment of hyperhidrosis. However, the usual duration of an intramuscular injection of a botulinum neurotoxin type A is typically about 3 to 4 months.

“Neurotoxin” means a biologically active molecule with a specific affinity for a neuronal cell surface receptor. Neurotoxin includes Clostridial toxins both as pure toxin and as complexed with one to more non-toxin, toxin-associated proteins.

“Patient” means a human or non-human subject receiving medical or veterinary care.

“Pharmaceutical composition” means a formulation in which an active ingredient can be a Clostridial toxin. The word “formulation” means that there is at least one additional ingredient (such as, for example and not limited to, an albumin [such as a human serum albumin or a recombinant human albumin] and/or sodium chloride) in the pharmaceutical composition in addition to a botulinum neurotoxin active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Therapeutic formulation” means a formulation that can be used to treat and thereby alleviate a disorder or a disease and/or symptom associated thereof.

“Therapeutically effective amount” means the level, amount or concentration of an agent (e.g. such as a clostridial toxin or pharmaceutical composition comprising clostridial toxin) needed to treat a disease, disorder or condition without causing significant negative or adverse side effects.

“Treat,” “treating,” or “treatment” means an alleviation or a reduction (which includes some reduction, a significant reduction, a near total reduction, and a total reduction), resolution or prevention (temporarily or permanently) of an symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX®)).

Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostidial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form; for example, it can be provided as a sterile solution in a vial or as a vial or sachet containing a lyophilized powder for reconstituting in a suitable vehicle such as saline for injection. In embodiments, the botulinum toxin is formulated in a solution containing saline and pasteurized Human Serum Albumin (HSA), which stabilizes the toxin and minimizes loss through non-specific adsorption. The solution can be sterile filtered (0.2 μm filter), filled into individual vials, and then vacuum-dried to give a sterile lyophilized powder. In use, the powder can be reconstituted by the addition of sterile unpreserved normal saline (sodium chloride 0.9% for injection).

In an embodiment, botulinum type A is supplied in a sterile solution for injection with a 5-mL vial nominal concentration of 1 ng/mL in 0.03 M sodium phosphate, 0.12 M sodium chloride, and 1 mg/mL HSA, at pH 6.0. Although a disclosed composition may only contain a single type of neurotoxin, for example botulinum type A, disclosed compositions can include two or more types of neurotoxins, which can provide enhanced therapeutic effects of the disorders. For example, a composition administered to a patient can include botulinum types A and E, or A and B, or the like. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, a quicker effect combined with a longer duration. The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABAA receptor. The GABAA receptor inhibits neuronal activity by effectively shunting current flow across the cell membrane. GABAA receptor modulators may enhance the inhibitory effects of the GABAA receptor and reduce electrical or chemical signal transmission from the neurons. Examples of GABAA receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors. Further disclosed compositions comprise esketamine.

Disclosed neurotoxin compositions can be injected into the patient using any suitable delivery system, for example, a needle or a needleless device. In certain embodiments, the method comprises injecting the composition into the SG or into the region adjacent to the SG. For example, administering may comprise injecting the composition through a needle of no greater than about 30 gauge. In certain embodiments, the method comprises administering a composition comprising a botulinum toxin type A.

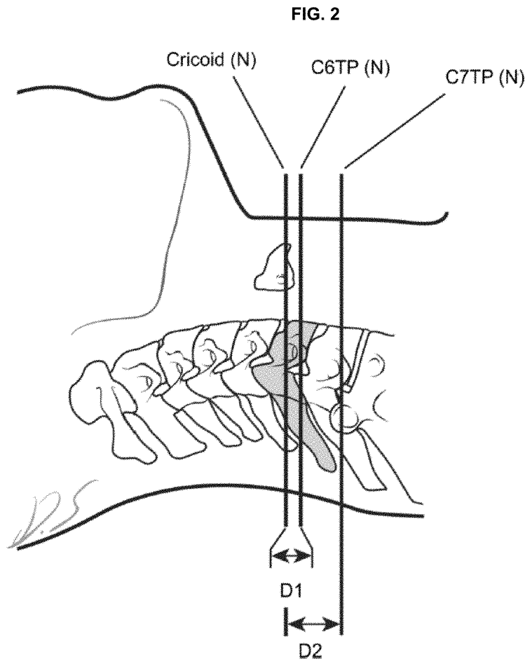

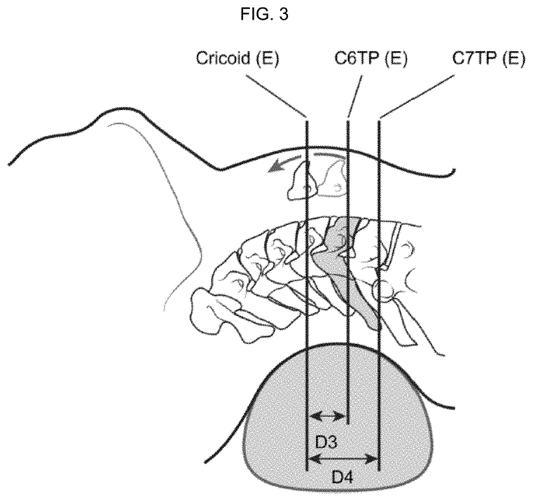

Administration of the disclosed compositions can be carried out by syringes, catheters, needles and other means for injecting. The injection can be performed on any area of the mammal's body that is in need of treatment, however disclosed embodiments contemplate injection into the base of the patient's neck, specifically into the SG or the region near the SG. The stellate ganglion (SG) is a collection of sympathetic nerves found at the level of the sixth and seventh cervical vertebrae (the last vertebra of the neck). The nerves are located in front of the vertebrae. There are superficial landmarks that can be used to identify the injection location, such as the Cricoid Cartilage of the Larynx. During the procedure either fluoroscopy imaging or ultrasound guidance can be used, however disclosed embodiments comprise administration without the use of imaging technologies. Fluoroscopy can be used to identify the bony landmarks and/or ultrasound guidance can be used to provide visualization of the arteries and veins in the neck without risk of exposure to radiation when administering the neurotoxin. The injection can be into any specific area such as a nerve junction, or area immediately adjacent to the SG. FIG. 2 shows a schematic of the neutral supine position of the neck. Cricoid(N) is the line perpendicular to the ground at the level of the middle point of the cricoid cartilage on the anterior neck surface in the neutral supine position. C6TP(N) is the line perpendicular to the neck and parallel to Cricoid(N), passing through the midpoint of the C6 transverse process in the anterolateral ultrasonographic view in the neutral supine position. C7TP(N) is the line perpendicular to the neck and parallel to the line Cricoid(N), passing through the midpoint of the C7 transverse process in the anterolateral ultrasonographic view in the neutral supine position. D1 is the distance between Cricoid(N) and C6TP(N). D2 is the distance between Cricoid(N) and C7TP(N). FIG. 3 shows Schematic of the extended supine position of the neck. Cricoid(E) is the line perpendicular to the ground at the level of the middle point of the cricoid cartilage on the anterior neck surface in the extended supine position. C6TP(E) is the line perpendicular to the neck and parallel to the line Cricoid(E), passing through the midpoint of the C6 transverse process in the anterolateral ultrasonographic view in the extended supine position. C7TP(E) is the line perpendicular to the neck and parallel to the line Cricoid(E), passing through the midpoint of the C7 transverse process in the anterolateral ultrasonographic view in the extended supine position. D3 is the distance between Cricoid(E) and C6TP(E). D4 is the distance between Cricoid(E) and C7TP(E). Further disclosure relating to the position of the SG can be found in, for example, Variations in the distance between the cricoid cartilage and targets of stellate ganglion block in neutral and extended supine positions: an ultrasonographic evaluation, J Anesth (2016) 30:999-1002 DOI 10.1007/s00540-016-2236-8, which is incorporated herein by reference in its entirety. FIG. 3 shows a schematic of the extended supine position of the neck. Cricoid(E) is the line perpendicular to the ground at the level of the middle point of the cricoid cartilage on the anterior neck surface in the extended supine position. C6TP(E) is the line perpendicular to the neck and parallel to the line Cricoid(E), passing through the midpoint of the C6 transverse process in the anterolateral ultrasonographic view in the extended supine position. C7TP(E) is the line perpendicular to the neck and parallel to the line Cricoid(E), passing through the midpoint of the C7 transverse process in the anterolateral ultrasonographic view in the extended supine position. D3 is the distance between Cricoid(E) and C6TP(E). D4 is the distance between Cricoid(E) and C7TP(E).

More than one injection and/or sites of injection may be necessary to achieve the desired result. Also, some injections, depending on the location to be injected, may require the use of fine, hollow, TEFLON®-coated needles. In certain embodiments, guided injection is employed, for example by electromyography, or ultrasound, or fluoroscopic guidance, or the like.

The frequency and the amount of injection using the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill). For example, the route and dosage for administration of a Clostridial neurotoxin according to the present disclosed invention can be selected based upon criteria such as the solubility characteristics of the neurotoxin chosen as well as the intensity and scope of the condition being treated.

Methods of Use

Methods disclosed herein can comprise administration of a neurotoxin, for example a Clostridial toxin, for example a botulinum type A, to the stellate ganglion of a patient, or in the vicinity thereof, to prevent, eliminate, or alleviate the symptoms associated with a cardiovascular disorder. For example, disclosed methods can prevent, reduce the occurrence of, or alleviate the occurrence of pain such as headache, chest pain including pressure, squeezing, burning, or fullness, difficulty breathing, irregular heartbeat, blood in the urine, pounding in the chest, neck, or ears, a feeling that the heart is skipping a beat or racing, or too slow, pauses between heartbeats, severe heart palpitations, anxiety, pressure, discomfort, shortness of breath, nausea, fatigue, vision problems, diminished exercise capability, swelling (edema), particularly swelling of the extremities, for example the lower extremities, heaviness, tightness, pain in the arms or shoulders, vomiting, back pain, jaw pain, lightheadedness, dizziness, fainting, irregular heartbeat, and combinations thereof.

Disorders suitable for treatment with disclosed methods comprise, for example, angina, arrhythmias, myocardial contractility disorder, coronary artery disease, cardiomyopathy, high blood pressure, and combinations thereof.

Symptoms of high blood pressure suitable for treatment with disclosed methods comprise severe headaches, fatigue, vision problems, difficulty breathing, irregular heartbeat, blood in the urine, and pounding in the chest, neck, or ears.

Symptoms of angina suitable for treatment with disclosed methods comprise chest pain or discomfort, possibly described as pressure, squeezing, burning or fullness, pain in the arms, neck, jaw, shoulder or back accompanying chest pain, nausea, fatigue, shortness of breath, sweating, and dizziness.

Symptoms of cardiac arrhythmias suitable for treatment with disclosed methods comprise a feeling that your heart is skipping a beat, a heartbeat that is too fast or “racing,” a heartbeat that is too slow, an irregular heartbeat, pauses between heartbeats, chest pain, shortness of breath, dizziness, lightheadedness, fainting or near-fainting, severe heart palpitations, anxiety, and sweating.

Symptoms of congestive heart failure suitable for treatment with disclosed methods comprise fatigue, diminished exercise capacity, shortness of breath, and swelling (edema).

Symptoms of coronary artery disease (CAD) suitable for treatment with disclosed methods comprise chest pain, heaviness, tightness, burning, and squeezing. These symptoms can also be mistaken for heartburn or indigestion. Other symptoms of CAD include pain in the arms or shoulders, shortness of breath, sweating, and dizziness. Women may be more likely to experience vomiting, back pain, jaw pain, and shortness of breath without feeling chest pain.

Disclosed embodiments can comprise the administration of at least one local anesthetic to the SG prior to administration of the neurotoxin, after administration of the neurotoxin or in combination with the neurotoxin administration. For example, 5 to 10 mL of a local anesthetic such as lidocaine 1 or 2% can be administered via injection to the SG, or to the vicinity of the SG. Suitable local anesthetics for use in disclosed embodiments include, for example, Ropivacaine, Bupivacaine, Mepivacaine, Chloroprocaine, Tetracaine, Tetracaine, Nesacaine-MPF, Lidocaine-MPF, Polocaine-MPF, and Sensorcaine-MPF, and the like.

Disclosed treatment methods comprise use of a neurotoxin after administration of a local anesthetic, prior to administration of a local anesthetic or in combination with a local anesthetic, with both applied to the SG or the vicinity thereof. Preferably, the local anesthetic is administered before the neurotoxin to numb the area prior to administration of the neurotoxin.

Disclosed methods can comprise administration of multiple clostridial neurotoxins.

Disclosed embodiments comprise establishing an SGB or modulating the activity of the SG and reducing the number or amount of other medications, for example cardiovascular disorder medication, prescribed to the patient.

Disclosed embodiments comprise administration of a neurotoxin to inhibit the release of gamma aminobutyric acid (GABA) or substance P, neuropeptides associated with pain.

Neurotoxin Dosages

The neurotoxin or neurotoxins can be administered in an amount of between about 10⁻³ U/kg and about 2 U/kg In an embodiment, the neurotoxin is administered in an amount of between about 10⁻² U/kg and about 1.5 U/kg. In another embodiment, the neurotoxin is administered in an amount of between about 10⁻¹ U/kg and about 0.5 U/kg. In many instances, an administration of from about 1 unit to about 120 Units of a neurotoxin, such as a botulinum type A, provides effective therapeutic relief. In an embodiment, from about 5 Units to about 100 Units of a neurotoxin, such as a botulinum type A, can be used and in another embodiment, from about 10 Units to about 100 Units of a neurotoxin, such as a botulinum type A, can be locally administered into a target tissue.

In embodiments, administration can comprise a total dose per treatment session of about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or the like.

In embodiments, administration can comprise a total dose per treatment session of not less than 10 Units of a neurotoxin, for example botulinum type A neurotoxin, or not less than 20 Units, or not less than 30 Units, or not less than 40 Units, or not less than 50 Units, or not less than 60 Units, or not less than 70 Units, or not less than 80 Units, or not less than 90 Units, or not less than 100 Units, or not less than 110 Units, or not less than 120 Units, or the like.

In embodiments, administration can comprise a total dose per treatment session of not more than 10 Units of a neurotoxin, for example botulinum type A neurotoxin, or not more than 20 Units, or not more than 30 Units, or not more than 40 Units, or not more than 50 Units, or not more than 60 Units, or not more than 70 Units, or not more than 80 Units, or not more than 90 Units, or not more than 100 Units, or not more than 110 Units, or not more than 120 Units, or the like.

In embodiments, administration can comprise a total dose per year of not more than 400 Units of a neurotoxin, for example botulinum type A neurotoxin, or not more than 500 Units, or not more than 600 Units, or not more than 700 Units, or not more than 800 Units, or not more than 900 Units, or not more than 1000 Units, or not more than 1100 Units, or not more than 1200 Units, or not more than 1300 Units, or not more than 1400 Units, or not more than 1500 Units, or not more than 1600 Units, or not more than 1700 Units, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Ultimately, however, both the quantity of toxin administered and the frequency of its administration will be at the discretion of the physician responsible for the treatment and will be commensurate with questions of safety and the effects produced by the toxin.

Disclosed embodiments comprise treatments that can be repeated. For example, a repeat treatment can be performed when the patient begins to experience symptoms associated with the neurologic and/or psychiatric disorder. However, preferred embodiments comprise repeating the treatment prior to the return of symptoms. Therefore, disclosed embodiments comprise repeating the treatment, for example, after 10 weeks, 12 weeks, 14 weeks, 16 weeks, 18 weeks, 20 weeks, 22 weeks, 24 weeks, or more. Repeat treatments can comprise administration sites that differ from the administration sites used in a prior treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

A kit for practicing disclosed embodiments is also encompassed by the present disclosure. The kit can comprise a 30 gauge or smaller needle and a corresponding syringe. The kit can also comprise a Clostridial neurotoxin composition, such as a botulinum type A toxin composition. The neurotoxin composition may be provided in the syringe. The composition is injectable through the needle. The kits are designed in various forms based the sizes of the syringe and the needles and the volume of the injectable composition(s) contained therein, which in turn are based on the specific deficiencies the kits are designed to treat.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of High Blood Pressure

A patient suffering from high blood pressure is treated via injection of 35 U of botulinum type A into the stellate ganglion (SG) to establish a stellate ganglion block (SGB) or modulate the activity of the SG. The patient lies on their back with a pillow placed under their shoulder blades. The patient's neck is cleansed with an antiseptic soap. The doctor counts the vertebrae of the spinal column to identify the correct cervical vertebrae C6 and C7 where the SG should be located. The SG can also be located superficially by locating the bony landmarks of the Cricoid Cartilage of the Larynx. Fluoroscopy and/or ultrasound imaging can then be used to identify the location of the SG when the patient is lying on his back to identify where to place the needle. The patient is asked to avoid talking, coughing, or swallowing, as these activities may cause the needle to move. The patient reports a reduction in their high blood pressure symptoms including chest pain, fatigue, vision problems, shortness of breath, irregular heartbeat, and sweating. This reduction lasts for 12 weeks.

### Example 2

### Treatment of Angina

An angina patient is treated via injection of 45 U of botulinum type A into the SG to establish an SGB or modulate the activity of the SG.

The patient reports a reduction in their angina symptoms including chest pain, arm pain, shoulder pain, back pain, shortness of breath, dizziness, and sweating. This reduction lasts for 16 weeks.

### Example 3

### Treatment of High Blood Pressure

A patient suffering from high blood pressure is treated via injection of 60 U of botulinum type B into the stellate ganglion (SG) to establish a stellate ganglion block (SGB) or modulate the activity of the SG. The patient is also administered amlodipine at a dose of 5 mg per day.

The patient reports a reduction in their high blood pressure symptoms including headache, chest pain, fatigue, vision problems, shortness of breath, irregular heartbeat, and blood in the urine. This reduction lasts for 10 weeks.

### Example 4

### Treatment of Arrhythmia

An arrhythmia patient is treated via injection of 40 U of botulinum type E as well as 2 mL of 1% lidocaine into the stellate ganglion (SG) to establish a stellate ganglion block (SGB) or modulate the activity of the SG. The patient is also administered a beta blocker.

The patient reports a reduction in their atrial and/or ventricular arrhythmia symptoms including feelings of the heart skipping a beat, racing, beating too slowly, and irregular heartbeat. This treatment is repeated after 6 weeks.

### Example 5

### Treatment of Coronary Artery Disease

A patient with a coronary artery disease is treated via injection of 100 U of botulinum type A into the stellate ganglion (SG) to establish a stellate ganglion block (SGB).

The patient reports a reduction in their symptoms associated with coronary artery disease (CAD) including, but not limited to: chest pain, pain in the arms or shoulders, shortness of breath, and sweating. This reduction lasts for 16 weeks.

### Example 6

### Treatment of Angina

An angina patient is treated via injection of 35 U of botulinum type B into the SG to establish an SGB or modulate the activity of the SG.

The patient reports a reduction in their angina symptoms including nausea, fatigue, sweating, shortness of breath, dizziness, chest pain such as pressure, squeezing, burning or fullness, and pain in the arms, neck, jaw, shoulder, or back. This reduction lasts for 10 weeks.

### Example 7

### Treatment of High Blood Pressure

A patient suffering from high blood pressure is treated via injection of 120 U of botulinum type A into the stellate ganglion (SG) to establish a stellate ganglion block (SGB). The patient is also administered chlorthalidone.

The patient reports a reduction in their high blood pressure symptoms including headache, chest pain, fatigue, vision problems, shortness of breath, irregular heartbeat, and blood in the urine. This reduction lasts for 13 weeks.

### Example 8

### Treatment of a Myocardial Contractility Disorder

A patient with a myocardial contractility disorder is treated via injection of 30 U of botulinum type A as well as 2 mL of 1% lidocaine into the stellate ganglion (SG) to establish a stellate ganglion block (SGB) or modulate the activity of the SG. The patient is also administered digoxin.

The patient reports a reduction in their symptoms associated with the myocardial contractility disorder including fatigue, diminished exercise capability, shortness of breath and edema. This treatment is repeated after 6 weeks.

### Example 9

### Treatment of Arrhythmia

An arrhythmia patient is treated via injection of 100 U of botulinum type B as well as 2 mL of 1% lidocaine into the stellate ganglion (SG) to establish a stellate ganglion block (SGB). The patient is also administered warfarin.

The patient reports a reduction in their arrhythmia symptoms including feelings of the heart skipping a beat, racing, too slow, or irregular. This treatment is repeated after 5 weeks.

### Example 10

### Treatment of High Blood Pressure

A patient suffering from high blood pressure is treated via injection of 70 U of botulinum type B into the stellate ganglion (SG) to establish a stellate ganglion block (SGB). The patient is also administered amlodipine.

The patient reports a reduction in their high blood pressure symptoms including headache, chest pain, fatigue, vision problems, shortness of breath, irregular heartbeat, and blood in the urine. This reduction lasts for 10 weeks.

### Example 11

### Treatment of High Blood Pressure

A patient suffering from high blood pressure is treated via injection of 100 U of botulinum type E into the stellate ganglion (SG) to establish a stellate ganglion block (SGB) or modulate the activity of the SG. The patient is also administered chlorthalidone.

The patient reports a reduction in their high blood pressure symptoms including headache, chest pain, fatigue, vision problems, shortness of breath, irregular heartbeat, and blood in the urine. This reduction lasts for 12 weeks.

### Example 12

### Treatment of Tachycardia

A patient suffering from tachycardia is treated via percutaneous injection of 40 U of botulinum type A into the stellate ganglion (SG) to establish a stellate ganglion block (SGB) or modulate the activity of the SG. This treatment follows the failure of previous treatment attempts using lidocaine.

The patient reports a reduction in their symptoms. This reduction lasts for 12-36 weeks.

### Example 13

### Treatment of Arrhythmia

A patient suffering from arrhythmia is treated via percutaneous injection of 85 U of botulinum type B into the stellate ganglion (SG) to establish a stellate ganglion block (SGB) or modulate the activity of the SG. This treatment follows the failure of previous treatment attempts using intravenous amiodarone.

The patient reports a reduction in their symptoms. This reduction lasts for 10-24 weeks.

### Example 14

### Treatment of Tachycardia

A patient suffering from tachycardia is treated via percutaneous injection of 120 U of botulinum type A into the stellate ganglion (SG) to establish a stellate ganglion block (SGB) or modulate the activity of the SG. The patient was also administered intravenous lidocaine.

The patient reports a reduction in their symptoms. This reduction lasts for 14-36 weeks.

### Example 15

### Treatment of Arrhythmia

A patient suffering from arrhythmia is treated via percutaneous injection of 55 U of botulinum type B into the stellate ganglion (SG) to establish a stellate ganglion block (SGB) or modulate the activity of the SG. The patient was also administered intravenous amiodarone.

The patient reports a reduction in their symptoms. This reduction lasts for 12-28 weeks.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

What is claimed is:

1)-38) (canceled)

39) A method of treating a cardiovascular disorder comprising:
administering a botulinum toxin into the stellate ganglion nerve of a mammal or the vicinity thereof to treat at least one of angina, arrhythmias of heart rate, a myocardial contractility disorder, coronary artery disease, and high blood pressure;
thereby reducing the occurrence of at least one symptom of the disorder.

40) The method of claim 39, wherein the botulinum toxin is native immunotype A.

41) The method of claim 40, wherein the botulinum toxin is administered by injection.

42) A method of treating a cardiovascular disorder comprising;
administering a botulinum toxin into the stellate ganglion or the vicinity thereof in a subject with a cardiovascular disorder to thereby treat their symptoms.

43) A method of treating a cardiovascular disorder comprising administering a botulinum toxin into the stellate ganglion or the vicinity thereof wherein the administration is performed by imaging the treatment area.

44) The method of claim 39 or 42-43, wherein the botulinum toxin is a botulinum toxin type A, B, C, E, or F.

45) The method of claim 44, wherein the total dose of the botulinum toxin is 50-100 Units.

46) The method of claim 44, wherein the total dose of the botulinum toxin is 25-120 Units.

47) The method of claim 44, wherein the total dose of the botulinum toxin is 40-120 Units.

48) The method of claim 45, wherein said cardiovascular disorder comprises arrhythmias of heart rate.

49) The method of claim 45, wherein said cardiovascular disorder comprises myocardial contractility.

50) The method of claim 45, wherein said cardiovascular disorder comprises a coronary artery disease.

51) The method of claim 45, wherein said cardiovascular disorder comprises high blood pressure.

52) A method for comparing the efficacy and safety of two different Botulinum toxins, comprising:
measuring a reduction of a cardiovascular disorder symptom of an individual resulting from administration of a first botulinum neurotoxin; measuring a reduction of a cardio disorder symptom of an individual resulting from administration of a second botulinum neurotoxin; and comparing the reduction in symptoms to determine a difference between the first botulinum neurotoxin and the second botulinum neurotoxin.

53) A method of treating a cardiovascular disorder comprising the steps of administering a botulinum toxin into the stellate ganglion nerve of a mammal or into the vicinity thereof to treat at least one of arrhythmias of heart rate, a myocardial contractility disorder, coronary artery disease, and high blood pressure; and administering a local anesthetic to the stellate ganglion nerve of said mammal, thereby reducing the occurrence of at least one symptom of the disorder.

54) The method of claim 53, wherein said local anesthetic comprises lidocaine.

55) The method of claim 53, wherein said botulinum toxin is type A, B, C, E or F.

56) The method of claim 1, wherein said administration comprises locating the cricoid cartilage of the larynx to identify the location of the stellate ganglion and injecting into the stellate ganglion or vicinity thereof.

57) The method of claim 43, wherein said imaging comprises fluoroscopy.

58) The method of claim 43, wherein said imaging comprises ultrasound.

59) The method of claim 57, wherein said treating arrhythmia comprises one of stabilizing ventricular rhythm or reducing tachycardia symptoms.

61) The method of claim 55, wherein the total dose of the botulinum toxin is 50-100 Units.

62) The method of claim 55, wherein the total dose of the botulinum toxin is 25-120 Units.

63) The method of claim 55, wherein the total dose of the botulinum toxin is 40-110 Units.

64) The method of claim 55, wherein said cardiovascular disorder comprises arrhythmias of heart rate.

65) The method of claim 55, wherein said cardiovascular disorder comprises myocardial contractility.

66) The method of claim 55, wherein said cardiovascular disorder comprises a coronary artery disease.

67) The method of claim 55, wherein said cardiovascular disorder comprises high blood pressure.

68) The method of claim 55, wherein said cardiovascular disorder comprises angina.
